(19) United States
(12) Patent Application Publication
(10) Pub. No.: US 2019/0183433 A1
(43) Pub. Date: Jun. 20, 2019

(54) METHOD, APPARATUS AND SYSTEM FOR GENERATING GATING SIGNAL OF MEDICAL IMAGING EQUIPMENT

(71) Applicant: SHENZHEN DARMA TECHNOLOGY CO., LTD., Shenzhen City (CN)

(72) Inventors: CHAO YANG, Shenzhen City (CN); JUNHAO HU, Shenzhen City (CN)

(21) Appl. No.: 16/194,400

(22) Filed: Nov. 19, 2018

(30) Foreign Application Priority Data
Sep. 19, 2017 (CN) 201710852209.4

(51) Int. Cl. A61B 5/00; A61B 5/11; A61B 5/113

(52) U.S. Cl. CPC A61B 5/7292; A61B 5/1102; A61B 5/1126; A61B 5/7278; A61B 5/113; A61B 5/7253; A61B 5/7203

(57) ABSTRACT

The present invention relates to medical equipments, and provides a method, apparatus and system for generating gating signal of medical imaging equipment. The method includes steps of: receiving optical signals obtained by an optical fiber sensor; controlling a photoelectric conversion unit to convert optical signals to electrical signals; extracting signals of BCG waveforms and/or respiration waveforms of the subject from the electrical signals; and generating gating signal of the medical imaging equipment based on the signals of BCG waveforms and/or respiration waveforms. Relative to the method of generating gate signal of the medical imaging equipment based on ECG waveforms, the method of the present invention does not need to a lead line and not need a doctor's work, which enables a better experience.

### FIELD OF THE INVENTION

The present invention relates to medical equipments, and especially relates to a method, apparatus and system for generating gating signal of a medical imaging equipment.

### BACKGROUND OF THE INVENTION

The medical equipments, such as MRI (Magnetic Resonance Imaging), CT (Computed Tomography), and PET-CT (Positron Emission Computed Tomography), are applied more and more widely in disease diagnosis due to many advantages such as being non-invasive, fast imaging, and high-resolution. For some structural heart diseases, tests such as MRI/CT are even the only diagnostic methods.

When MM equipment is used to image the heart, one RF Pulse Sequence will generate extremely low signal-to-noise ratio of MR (Magnetic resonance) signal, thus unable to rebuild clear images. So, it is necessary to use scanning technique to repeatedly transmit a series of RF pulse sequences and repeatedly collect signals for a longer scanning time, in such way that a graph can be received by superimposed reconstruction. If the imaging object cannot remain still during the scanning process, each scan will appear randomly at different times of the cardiac cycle, and the received MR signals will be from different states in the cardiac process, therefore, the heart image obtained from multiple non-homomorphic signals have a serious motion artifact.

The artifact problem can be solved by keeping the imaging object still. CT has short scan time, and it only takes tens of millisecond via ultra-high speed scanning, thus the motion artifact can avoid via holding breath when scanning a subject such as scanning his chest. However, MRI and PET-CT equipment have long scanning time, and it usually takes more than ten minutes, thereby the subject to be scanned cannot hold his breath or heartbeat. At present, an effective way, that is to keep both transmitting RF pulse sequences and collecting MR signals via MRI equipment being synchronized with the motion of the organ to be scanned. For example, scanning the heart can be synchronized with cardiac motion, that is, scanning can be performed at the same time in each cardiac cycle. Therefore, real-time monitoring of heartbeat and respiration is required for both MRI and PET-CT scans, gating signals are generated when imaging condition is met, and the medical imaging equipment starts to scan the motion organs such as the heart once receives the gating signal.

The existing method for generating gating signal of a medical imaging equipment based on electro cardiogram (ECG) signals or blood oxygen signals, which needs to attach a lead line to the body of the patient, thereby, it needs a doctor's work, and the patient's experience is also poor.

### SUMMARY OF THE INVENTION

The objective of the present invention is to provide a method, apparatus and system for generating gating signal of a medical imaging equipment, which aims to solve the problems that the existing method for generating gating signal of the medical imaging equipment based on electro cardiogram (ECG) signals or blood oxygen signals needs to attach a lead line to the body of the patient and needs a doctor's work, and the patient's experience is also poor.

A first aspect of the invention is to provide a method for generating gating signal of a medical imaging equipment, comprising steps of:

receiving optical signals obtained by an optical fiber sensor;

controlling a photoelectric conversion unit to convert optical signals to electrical signals;

extracting signals of BCG waveforms and/or respiration waveforms of the subject from the electrical signals; and

generating gating signal of the medical imaging equipment based on the signals of BCG waveforms and/or respiration waveforms.

A second aspect of the invention is to provide an apparatus for generating gating signal of a medical imaging equipment, comprises:

a receiving module, for receiving optical signals obtained by an optical fiber sensor;

a control module, for controlling a photoelectric conversion unit to convert optical signals to electrical signals;

an extracting module, for extracting signals of BCG waveforms and/or respiration waveforms of the subject from the electrical signals; and

a gating-signal generation module, for generating gating signal of the medical imaging equipment based on the signals of BCG waveforms and/or respiration waveforms.

A third aspect of the present invention is to provide a computer-readable storage medium on which a computer program is stored, the program is executed by a processor to perform steps of the above method for generating gating signal of the medical imaging equipment.

A fourth aspect of the present invention is to provide a system for generating gating signal of a medical imaging equipment, comprising an optical fiber sensor, and a signal processing device for generating gating signal of the medical imaging equipment, the signal processing device is connected with the optical fiber sensor via an optical fiber and connected with a medical imaging equipment. Wherein:

the signal processing device comprises one or more processors, a memory, and one or more computer programs;
the one or more computer programs are stored on the memory and are configured to be executed by the one or more processors to perform steps of the above method for generating gating signal of the medical imaging equipment.

In the method of the present invention, detecting optical signals by the optical fiber sensor, extracting signals of BCG waveforms and/or respiration waveforms of the subject, and generating gating signal of the medical imaging equipment based on signals of BCG waveforms and/or respiration waveforms. Relative to the method of generating gate signal of the medical imaging equipment based on ECG waveforms, the method of the present invention does not need to attach a lead line to the body of the subject and not need a doctor's work as well, which enables a better experience. Furthermore, because the transmission of optical signals is not affected by magnetic field or radiation, and signals of BCG waveforms and/or respiration waveforms are detected using the optical fiber sensor; thus connecting cables do not need shielding design, which makes hardware design simpler and cost less.

### BRIEF DESCRIPTION OF THE DRAWINGS

FIG. 1 is a flowchart illustrating a method of generating gating signal of a medical imaging equipment in accordance with a first embodiment of the present invention;

FIG. 2 illustrates a waveform generated from optical signals obtained by an optical fiber sensor after photoelectric conversion;

FIG. 3 illustrates a time-domain diagram of extracted BCG waveform signals;

FIG. 4 illustrates a time-domain diagram of extracted respiration waveform signals;

FIG. 5 illustrates a cardiac cycle waveform;

FIG. 6 is a block diagram illustrating an apparatus for generating gating signal of a medical imaging equipment in accordance with a second embodiment of the present invention; and

FIG. 7 is a block diagram illustrating a system for generating gating signal of a medical imaging equipment in accordance with a forth embodiment of the present invention.

### DETAILED DESCRIPTION

Aspects, features, and advantages of the present invention will become clearer and better understood by reference to the following detailed description of the embodiments of the invention when considered in connection with the accompanying drawings. The following description of certain embodiments of the invention are not intended to limit the invention to these embodiments, but rather, are intended only to explain the present invention.

The features of the present will now be described in connection with the following detailed embodiments.

### First Embodiment

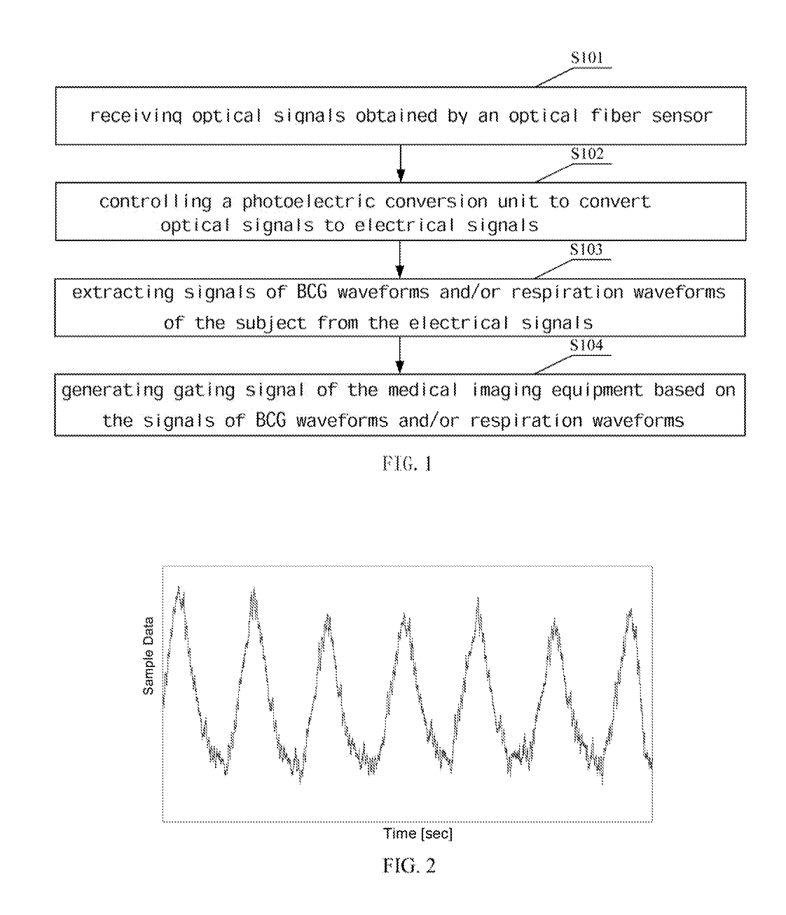

Referring to FIG. 1, a method for generating gating signal of a medical imaging equipment in accordance with the first embodiment, comprises steps described below: if there are essentially the same results, the method for generating gating signal of the medical imaging equipment of the present invention should not be limited to performs the steps in the order illustrated in FIG. 1.

S101, receiving optical signals obtained by an optical fiber sensor.

S101 in accordance with the first embodiment, specifically is:

controlling an optical fiber sensor to obtain optical signals, and then receiving optical signals from the optical fiber sensor;

### Or,

receiving optical signals from the optical fiber sensor, and the optical signals being actively obtained by the optical fiber sensor.

The optical fiber sensor can be arranged in a cushion, placed under a bed and positioned below the shoulder of the subject. When the subject lies on the bed, both his breath and heartbeat can cause subtle vibrations of the body, which leads to a subtle change of pressure applied on the optical fiber sensor. The change of the applied pressure causes changes of the light signals transmitted in the optical fiber sensor, and the optical fiber sensor can capture the changes of the optical signals. The optical fiber sensor can continuously detect the optical signals, and may start from the subject to be scanned lying on the bed and until a medical imaging equipment finishes imaging the subject.

S102, controlling a photoelectric conversion unit to convert optical signals to electrical signals.

As shown in FIG. 2, a waveform is generated from optical signals obtained by the optical fiber sensor after photoelectric conversion, where the outline of the signals are respiratory signal envelopes produced by respiration of a human subject, and while heartbeat and other interfering noise are combined to the curves of the respiratory signal envelopes.

S103, extracting signals of Ballistocardiogram (BCG) waveforms and/or respiration waveforms from the electrical signals.

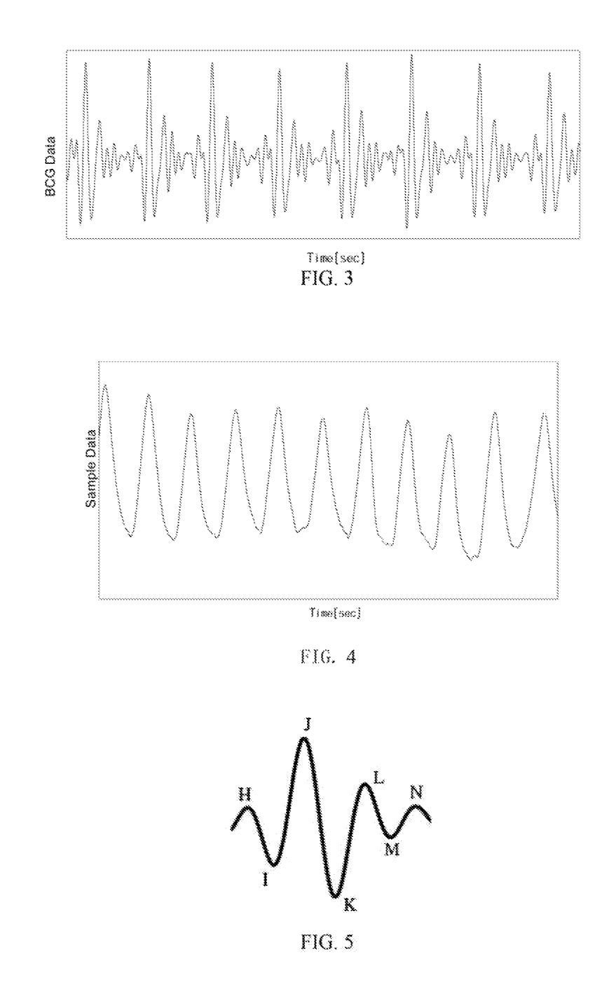

In accordance with the first embodiment of the present invention, S103 specifically is: extracting signals of Ballistocardiogram waveforms (BCG) and/or respiration waveforms after pre-filtering, amplifying, analog-to-digital conversion, sampling, filtering and de-noising, and signal scaling for electrical signals. As shown in FIG. 3, a time-domain diagram for extracted BCG waveform signals is illustrated. As shown in FIG. 4, a time-domain diagram for extracted respiration waveform signals is illustrated.

S104, generating gating signal of the medical imaging equipment based on the signals of BCG waveforms and/or respiration waveforms.

Herein, the step of generating gating signal of a medical imaging equipment based on the signals of BCG waveforms, specifically is:

based on the correspondence between examined diseases and/or changes in movement of the scanned organ (such as cardiac mechanical movement) and BCG waveforms, generating gating signals when a wave corresponding to the examined diseases and/or changes in movement of the scanned organ is detected.

In accordance with the first embodiment of the present invention, the wave corresponding to the examined diseases and/or changes in movement of the scanned organ, can be detected via detecting the time-domain signals of BCG waveforms, or via detecting the frequency-domain signals of BCG waveforms after being transformed from the time-domain signals of BCG waveforms by a time-frequency domain transformation.

One complete BCG waveform, namely a cardiac-cycle waveform as shown in FIG. 5, mainly comprises an H-peak, I-peak, J-peak, K-peak, M-peak and N-peak. It is generally believed that H-peak, I-peak, J-peak and K-peak are related to cardiac contraction, while M-peak and N-peak are related to cardiac relaxation. For example, gating signal can be generated when J-peak is detected, or when M-peak is detected.

In accordance with the first embodiment of the present invention, derivation calculation, bubble calculation method, or other calculation methods can be used in the detection of J-peak.

In accordance with the first embodiment of the present invention, the step of generating gating signal of the medical imaging equipment based on the signals of respiration waveforms, specifically is:

based on the correspondence between examined diseases and/or changes in movement of the scanned organ (such as movement of organs in the thoracic and abdominal cavity) and respiration waveforms, generating gating signals when a wave corresponding to the examined diseases and/or changes in movement of the scanned organ is detected.

In accordance with the first embodiment of the present invention, the wave corresponding to the examined diseases and/or changes in movement of the scanned organ, can be detected via detecting the time-domain signals of respiration waveforms, or via detecting frequency-domain signals of respiration waveforms after being transformed from the time-domain signals of respiration waveforms by a time-frequency domain transformation. For example, gating signal can be generated when the maximum peak in a respiration cycle is detected; or gating signal can be generated when the minimum valley in a respiration cycle is detected.

In accordance with the first embodiment of the present invention, derivation calculation, bubble calculation method, or other calculation methods can be used in detecting the maximum peak of the respiration wave.

### Second Embodiment

Referring to FIG. 6, an apparatus for generating gating signal of a medical imaging equipment in accordance with the second embodiment, comprises:

a receiving module 11, for receiving optical signals obtained by an optical fiber sensor;

a control module 12, for controlling a photoelectric conversion unit to convert optical signals to electrical signals;

an extracting module 13, for extracting signals of BCG waveforms and/or respiration waveforms of the subject from the electrical signals; and

a gating-signal generation module 14, for generating gating signal of the medical imaging equipment based on the signals of BCG waveforms and/or respiration waveforms.

### Third Embodiment

In accordance with the third embodiment of the present invention, a computer-readable storage medium on which a computer program is stored, the program is executed by a processor to perform steps of the method for generating gating signal of the medical imaging equipment in the first embodiment of the present invention.

### Fourth Embodiment

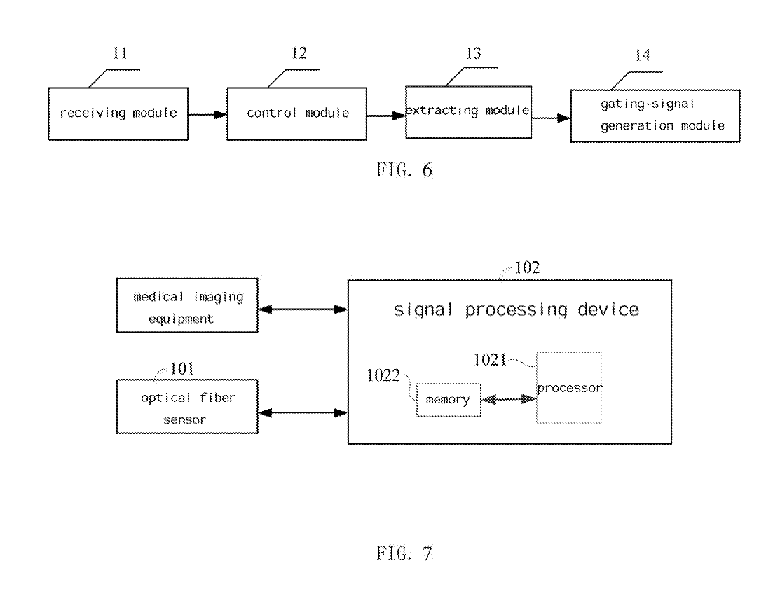

FIG. 7 illustrates a detailed block diagram of a system for generating gating signal of a medical imaging equipment in accordance with the forth embodiment of the present invention. The system for generating gating signal of the medical imaging equipment comprises: an optical fiber sensor 101, and a signal processing device 102 for generating gating signal of the medical imaging equipment. The signal processing device 102 is connected with the optical fiber sensor via an optical fiber and connected with the medical imaging equipment. The signal processing device 102 comprises one or more processors 1021, a memory 1022, and one or more computer programs. The one or more computer programs are stored on the memory 1022 and are configured to be executed by the one or more processors 1021. The processors 1021 execute the computer programs to perform steps of the method for generating gating signal of the medical imaging equipment in the first embodiment of the present invention.

In accordance with the fourth embodiment, the signal processing device can be a separate device, such as a PC, which is placed in a control room, and is connected with the medical imaging equipment. The connection may be wired, including but not limited to, communication bus such as I2C, SPI, UART, CAN, USB, PCIE; or the connection may be wireless, including but not limited to, WIFI, Bluetooth, Zigbee and etc. Via such configuration, gating signal generated by the signal processing device is transmitted to a computer system of the medical imaging equipment; after received the gating signal, the computer system can control to start a scan device of the medical imaging equipment to scan the subject.

The signal processing device can also be integrated in the scan device or a computer system of the medical imaging equipment; for example, the signal processing device can be an integrated circuit board, including core processors such as MCU, DSP, FPGA etc.

Both the optical fiber sensor and the signal processing device can also be integrated in the medical imaging equipment; for example, the optical fiber sensor can be integrated in the bed of the medical imaging equipment, and the signal processing device is integrated in the scan device or the computer system of the medical imaging equipment.

When the signal processing device is integrated in the scan device of the medical imaging equipment, the optical fiber connected between the optical fiber sensor and the signal processing device can also be integrated in the scan device of the medical imaging equipment.

In the method provided in accordance with the embodiments of the present invention, detecting optical signals by the optical fiber sensor, extracting signals of BCG waveforms and/or respiration waveforms of the subject, and generating gating signal of the medical imaging equipment based on signals of BCG waveforms and/or respiration waveforms. Relative to the method of generating gate signal of the medical imaging equipment based on ECG waveforms, the method of the present invention does not need to attach a lead line to the body of the subject and not need a doctor's work as well, which enables a better experience. Furthermore, because the transmission of optical signals is not affected by magnetic field or radiation, and signals of BCG waveforms and/or respiration waveforms are detected using the optical fiber sensor; thus connecting cables do not need shielding design, which makes hardware design simpler and cost less.

The above mentioned is preferred embodiments of the invention and is not used to limit the invention. Any modification, equivalent replacement and improvement made within the spirit and principles of the invention, shall be included in the protection scope of the invention.

1. A method for generating gating signal of a medical imaging equipment, comprising steps of:
receiving optical signals obtained by an optical fiber sensor;
controlling a photoelectric conversion unit to convert optical signals to electrical signals;
extracting signals of BCG waveforms and/or respiration waveforms of the subject from the electrical signals; and
generating gating signal of the medical imaging equipment based on the signals of BCG waveforms and/or respiration waveforms.

2. The method of claim 1, wherein the step of receiving optical signals obtained by an optical fiber sensor is:
controlling the optical fiber sensor to obtain optical signals, and then receiving optical signals from the optical fiber sensor; or
receiving optical signals from the optical fiber sensor, and the optical signals being actively obtained by the optical fiber sensor;
the step of extracting signals of BCG waveforms and/or respiration waveforms of the subject from the electrical signals is:
extracting signals of BCG waveforms and/or respiration waveforms after pre-filtering, amplifying, analog-to-digital conversion, sampling, filtering and de-noising, and signal scaling for electrical signals.

3. The method of claim 1, wherein the step of generating gating signal of the medical imaging equipment based on the signals of BCG waveforms is:
based on a correspondence between examined diseases and/or changes in movement of a scanned organ and BCG waveforms, generating gating signals when a wave corresponding to the examined diseases and/or changes in movement of the scanned organ is detected.

4. The method of claim 3, wherein the wave corresponding to the examined diseases and/or changes in movement of the scanned organ is detected via detecting time-domain signals of BCG waveforms; or via detecting frequency-domain signals of BCG waveforms after being transformed from the time-domain signals of BCG waveforms by a time-frequency domain transformation.

5. The method of claim 1, wherein the step of generating gating signal of the medical imaging equipment based on the signals of respiration waveforms is:
based on a correspondence between examined diseases and/or changes in movement of a scanned organ and respiration waveforms, generating gating signals when a wave corresponding to the examined diseases and/or changes in movement of the scanned organ is detected.

6. The method of claim 5, wherein the wave corresponding to the examined diseases and/or changes in movement of the scanned organ, is detected via detecting time-domain signals of respiration waveforms, or via detecting frequency-domain signals of respiration waveforms after being transformed from the time-domain signals of respiration waveforms by a time-frequency domain transformation.

7. (canceled)

8. A computer-readable storage medium, on which a computer program is stored, wherein the program is executed by a processor to perform a method for generating gating signal of the medical imaging equipment, the method comprising steps of:
receiving optical signals obtained by an optical fiber sensor;
controlling a photoelectric conversion unit to convert optical signals to electrical signals;
extracting signals of BCG waveforms and/or respiration waveforms of the subject from the electrical signals; and
generating gating signal of the medical imaging equipment based on the signals of BCG waveforms and/or respiration waveforms.

9. A system for generating gating signal of a medical imaging equipment, comprising:
an optical fiber sensor, and a signal processing device for generating gating signal of the medical imaging equipment, the signal processing device being connected with the optical fiber sensor via an optical fiber and connected with a medical imaging equipment, wherein:
the signal processing device comprises one or more processors, a memory, and one or more computer programs;
the one or more computer programs are stored on the memory and are configured to be executed by the one or more processors to perform steps of a method of for generating gating signal of the medical imaging equipment, the method comprising steps of:
receiving optical signals obtained by an optical fiber sensor;
controlling a photoelectric conversion unit to convert optical signals to electrical signals;
extracting signals of BCG waveforms and/or respiration waveforms of the subject from the electrical signals; and
generating gating signal of the medical imaging equipment based on the signals of BCG waveforms and/or respiration waveforms.

10. The system of claim 9, wherein the signal processing device is integrated in a scan device or a computer system of the medical imaging equipment; or, both the optical fiber sensor and the signal processing device are integrated in the medical imaging equipment.

11. The system of claim 9, wherein the step of receiving optical signals obtained by an optical fiber sensor is:
controlling the optical fiber sensor to obtain optical signals, and then receiving optical signals from the optical fiber sensor; or
receiving optical signals from the optical fiber sensor, and the optical signals being actively obtained by the optical fiber sensor;
the step of extracting signals of BCG waveforms and/or respiration waveforms of the subject from the electrical signals is:
extracting signals of BCG waveforms and/or respiration waveforms after pre-filtering, amplifying, analog-to-digital conversion, sampling, filtering and de-noising, and signal scaling for electrical signals.

12. The system of claim 9, wherein the step of generating gating signal of the medical imaging equipment based on the signals of BCG waveforms is:
based on a correspondence between examined diseases and/or changes in movement of a scanned organ and BCG waveforms, generating gating signals when a wave corresponding to the examined diseases and/or changes in movement of the scanned organ is detected.

13. The system of claim 12, wherein the wave corresponding to the examined diseases and/or changes in movement of the scanned organ is detected via detecting time-domain signals of BCG waveforms; or via detecting frequency-domain signals of BCG waveforms after being transformed from the time-domain signals of BCG waveforms by a time-frequency domain transformation.

14. The system of claim 9, wherein the step of generating gating signal of the medical imaging equipment based on the signals of respiration waveforms is:
based on a correspondence between examined diseases and/or changes in movement of a scanned organ and respiration waveforms, generating gating signals when a wave corresponding to the examined diseases and/or changes in movement of the scanned organ is detected.

15. The system of claim 14, wherein the wave corresponding to the examined diseases and/or changes in movement of the scanned organ, is detected via detecting time-domain signals of respiration waveforms, or via detecting frequency-domain signals of respiration waveforms after being transformed from the time-domain signals of respiration waveforms by a time-frequency domain transformation.

16. The computer-readable storage medium of claim 8, wherein the step of receiving optical signals obtained by an optical fiber sensor is:
controlling the optical fiber sensor to obtain optical signals, and then receiving optical signals from the optical fiber sensor; or
receiving optical signals from the optical fiber sensor, and the optical signals being actively obtained by the optical fiber sensor;
the step of extracting signals of BCG waveforms and/or respiration waveforms of the subject from the electrical signals is:
extracting signals of BCG waveforms and/or respiration waveforms after pre-filtering, amplifying, analog-to-digital conversion, sampling, filtering and de-noising, and signal scaling for electrical signals.

17. The computer-readable storage medium of claim 8, wherein the step of generating gating signal of the medical imaging equipment based on the signals of BCG waveforms is:
based on a correspondence between examined diseases and/or changes in movement of a scanned organ and BCG waveforms, generating gating signals when a wave corresponding to the examined diseases and/or changes in movement of the scanned organ is detected.

18. The computer-readable storage medium of claim 17, wherein the wave corresponding to the examined diseases and/or changes in movement of the scanned organ is detected via detecting time-domain signals of BCG waveforms; or via detecting frequency-domain signals of BCG waveforms after being transformed from the time-domain signals of BCG waveforms by a time-frequency domain transformation.

19. The computer-readable storage medium of claim 8, wherein the step of generating gating signal of the medical imaging equipment based on the signals of respiration waveforms is:
based on a correspondence between examined diseases and/or changes in movement of a scanned organ and respiration waveforms, generating gating signals when a wave corresponding to the examined diseases and/or changes in movement of the scanned organ is detected.

20. The computer-readable storage medium of claim 19, wherein the wave corresponding to the examined diseases and/or changes in movement of the scanned organ, is detected via detecting time-domain signals of respiration waveforms, or via detecting frequency-domain signals of respiration waveforms after being transformed from the time-domain signals of respiration waveforms by a time-frequency domain transformation.
